(19) United States
(12) Patent Application Publication
(10) Pub. No.: US 2025/0332231 A1
(43) Pub. Date: Oct. 30, 2025

(54) TREATING CANCER WITH CAS ENDONUCLEASE COMPLEXES

(71) Applicant: Stitch Bio, LLC, Waltham, MA (US)

(72) Inventor: Anthony P. Shuber, Northbridge, MA (US)

(21) Appl. No.: 18/972,013

(22) Filed: Dec. 6, 2024

Related U.S. Application Data
(63) Continuation of application No. 18/642,413, filed on Apr. 22, 2024, now abandoned.
Application No. 18/642,413 is a continuation of application No. 18/367,676, filed on Sep. 13, 2023, now abandoned.
Application No. 18/367,676 is a continuation of application No. 16/818,211, filed on Mar. 13, 2020, now abandoned.
Application No. 16/818,211 is a continuation-in-part of application No. 15/927,040, filed on Mar. 20, 2018, now abandoned.
(60) Provisional application No. 62/474,149, filed on Mar. 21, 2017.
(60) Provisional application No. 62/474,149, filed on Mar. 21, 2017.

(51) Int. Cl. A61K 38/46; C12N 15/11

(52) U.S. Cl. CPC A61K 38/465; C12N 15/11; C12N 2310/20; C12N 2320/30

(57) ABSTRACT

The invention generally relates to compositions and methods for targeted delivery of a Cas endonuclease or nucleic acid encoding a Cas endonuclease to a fusion sequence in a cancer cell but not in a healthy cell of a subject. The Cas endonuclease or nucleic acid encoding the Cas endonuclease may be complexed with a guide RNA complementary to a fusion sequence identified based on differences between a mutated sequence obtained from a cancer cell and a wild-type sequence obtained from a healthy cell of the subject. For example, the Cas endonuclease may be a Cas9 and cut DNA or a Cas13a and cut RNA. The Cas endonuclease complexes may induce cell death or cancerous cells or cause other beneficial effects.

### CROSS-REFERENCE TO RELATED APPLICATION

This application is a continuation in part of U.S. Non-Provisional application Ser. No. 15/927,040, which was filed Mar. 20, 2018, which claims priority to U.S. Provisional Patent Application No. 62/474,149, filed Mar. 21, 2017, the entire contents of which are incorporated by reference herein in their entirety.

### TECHNICAL FIELD

The disclosure relates to compositions and methods for treating cancer with Cas endonuclease complexes.

### BACKGROUND

A variety of therapies are available for treatment of cancer in a subject, including drug treatment therapy, radiation therapy, surgery, and alternative therapies. Often, these therapies act by killing cells of the body that divide rapidly, such as cancerous cells, but also normal cells such as hair follicles, cells of the digestive tract, and bone marrow. Thus, a problem with those therapies is that they are non-specific for targeting a cancerous cell because such therapies kill normal and cancerous cells. While killing the cancerous cells, collateral damage and death to the normal cells typically results in other deleterious effects to the patient, for example, loss of hair, blood disorders such as leucopenia and thrombocytopenia, digestive disorders, and physical pain.

### SUMMARY

This disclosure provides compositions and methods for targeted delivery of a Clustered Regularly Interspaced Short Palindromic Repeats (“CRISPR”) associated protein, or “Cas” endonuclease or nucleic acid encoding a Cas endonuclease to a fusion sequence present in a cancer cell or pre-cancerous cell to treat cancer in a subject. The Cas endonuclease or nucleic acid encoding a Cas endonuclease may be complexed with a guide RNA to allow the complex to target the fusion sequence. The fusion sequences targeted may be present in cancer or pre-cancerous cells but not in healthy cells.

Through the use of a guide RNA complementary to the identified fusion sequence, the Cas endonuclease complexes may be specifically and meaningfully directed to desired locations of a genome. This specificity allows the Cas endonucleases to perform beneficial functions, such as inducing cell death of cancerous or pre-cancerous cells, while minimizing deleterious effects to the subject. The guide RNA's complexed with Cas endonucleases may be created based on differences identified between a mutated sequence obtained from a cancer cell and a wild-type sequence obtained from a healthy cell of the subject.

Cancers typically result from genomic instability, for instance, a disruption in genomic stability, such as a mutation, that has been linked to the onset or progression of a cancer. A typical mutation event that gives rise to a cancer or a pre-cancerous cell is a loss of genetic material from a wild-type sequence, e.g., a deletion event. Thus a mutated sequence from a cancerous or pre-cancerous cell from a subject is typically missing a region of genomic material compared to a wild-type sequence from a normal cell. The disclosed compositions and methods take advantage of those sequence differences between a subject's normal healthy cells and those that are cancerous or pre-cancerous for treatment of cancer in the subject by specifically targeting and killing the diseased cells.

The disclosed methods involve introducing a Cas endonuclease complex that induces cell death in cells having genomic instability, but that is typically inert in wild-type cells. The disclosed compositions and methods selectively target genomic instability and, thus, selectively target cancer cells. The disclosed compositions may selectively kill cancer cells while not damaging healthy cells (i.e., cells that do not contain genomic instability). As a result, side effects of treatment are significantly reduced, along with a reduction in the impairment of normal tissue function. An embodiment of the disclosed composition includes a CRISPR/Cas9 complex that induces cell death in genomically-unstable cells, but that does not kill healthy cells.

Generally, Cas endonucleases are proteins involved in both cellular apoptosis and proliferation. Any Cas endonuclease may be used in the disclosed methods and compositions. The guide RNA may be covalently linked to the Cas endonuclease. For example, the Cas endonuclease may be a Cas9 and cut DNA. This complex including a Cas9 endonuclease may be referred to as a CRISPR/Cas9 complex. In another example, the Cas endonuclease may be a Cas13a and cut RNA. This complex including the Cas13a endonuclease may be referred to as a CRISPR/Cas13a complex.

It is understood that although certain exemplary embodiments are described as relating to CRISPR/Cas9 complexes that may induce cell death or other effects by acting upon DNA, that other similar embodiments relating to CRISPR/Cas13a complexes may induce cell death and other similar effects in RNA. This disclosure relates generally to Cas endonuclease complexes that may induce cell death or other beneficial effects and encompasses use of any Cas endonuclease.

An exemplary embodiment of the disclosed composition includes a CRISPR/Cas9 complex whereby the CRISPR/Cas9 targets a unique DNA sequence fusion sequence in genomically unstable cells, wherein the target regions are not present in healthy cells. One embodiment of the disclosed composition includes a CRISPR/Cas9 complex whose guide RNA template will hybridize to a fusion sequence present in the genomic DNA of a cancer cell that is not present in a healthy cell. The CRISPR/Cas9 complex has associated with it a guide RNA complementary to a Chromosome Instability (“CIN”) associated fusion sequence identified within the cancer cells, and has the capability to cut the genomic DNA strand at the site of the complementary fusion sequence therefore inducing cell death. The CRISPR/Cas9 complex may only create a double strand break at the site where the CIN associated fusion sequence is present and the guide RNA molecules are complementary. The guide RNA sequence within the CRISPR/Cas9 complex is designed to hybridize only to the region of the target genome that contain fusion sequences, and that are not present in the DNA of normal cells. The design of the guide RNA is preferably, but not necessarily, driven by sequencing nucleic acid in cancer cells (e.g., cells from a biopsy) to determine where genomic instability (e.g., a deletion) has occurred.

Methods are contemplated which include administering a mixture of CRISPR/Cas9 complexes to a subject. Each CRISPR/Cas9 complex within the treatment mixture contains a guide RNA molecule that will only recognize and bind with its complementary fusion sequence uniquely present in the genomic DNA of the cancer cells.

Creating and administering a mixture of CRISPR/Cas9 complexes also is advantageous due to the fact that not all cancer associated fusion sequences identified within the cancer genome will have the appropriate Protospacer Adjacent Motif (“PAM”) recognition site necessary for the CRISPR/Cas9 complexes recognition and the RNA/DNA base pairing. By creating a mixture of CRISPR/Cas9 complexes, it will increase the statistical likelihood of utilizing one if not more of the complexes to kill the cancer cell by cleaving the genomic DNA at several regions within the cancer cell and therefore inducing cell death.

Another advantage to administering a mixture of CRISPR/Cas9 complexes is to reduce potential drug toxicity. It has been recognized that CRISPR/Cas9 sequence recognition is not perfect, and that there is an associated “off rate” which involves the CRISPR/Cas9 complex to interact with other non-specific regions within the genome. The rate of non-homologous interaction of CRISPR/Cas9 complexes within normal cells of a patient may result in a certain level of normal cell death. By utilizing a mixture of CRISPR/Cas9 complexes, it will be possible to significantly reduce concentration of each CRISPR/Cas9 and therefore reduce the level of non-specific interaction of each CRISPR/Cas9 complex. Efficacy of the CRISPR/Cas9 complex mixture will then be determined by targeting a few or several CIN associated fusion sequences identified within the cancer cells.

For example, after identifying several CIN associated fusion sequences present in a patient's cancer cells that are not present in their normal cells, CRISPR/Cas9 complexes may be designed with guide RNA sequences complementary to the cancer specific fusion sequences that also have PAM recognition sites. Upon administration of the CRISPR/Cas9 complexes to a subject the CRISPR/Cas9 complexes each target their respective fusion sequences and create several double strand cuts within the cancer genome, therefore inducing cell death within the cancer cells. However, due to the lack of homology within the normal healthy cells, the CRISPR/Cas9 complexes do not interact with the genomic DNA, and the normal cells are unharmed.

A second embodiment involves utilizing CRISPR/Cas9 complexes to introduce a novel gene sequence into the cancer specific fusion sites that is expressed utilizing the normal cell mechanisms of expression. As with the first embodiment, the CRISPR/Cas9 complexes would recognize the cancer specific fusion sequences not present within the normal cells. However, whereas the first embodiment disclosed relied on multiple site specific double strand cuts of the genomic DNA initiating the endogenous cell death mechanism, the second embodiment disclosed introduces a gene sequence that results in the expression of a novel and lethal protein product. As with the first embodiment, a single CRISPR/Cas9 complex or a mixture of CRISPR/Cas9 complexes can be utilized to introduce the lethal protein associated gene sequence. In addition, by utilizing a mixture of CRISPR/Cas9 complexes, toxicity due to “non-specific” interaction of the CRISPR/Cas9 complexes within the genome can be limited by having the lethal level of protein expressed being associated with the sum of the fusion sequences and not a single site of novel gene introduction.

A third embodiment involves the introduction of a novel gene sequence that would express a marker cell surface antigen only associated with cancer cells and not present on the cell surface of normal healthy cells not containing the fusion sequence within their genome. With this embodiment, a single marker cell surface antigen associated cancer therapeutic could be utilized for cancer treatment regardless of any patient or tumor specific “driver” mutation. This approach would also allow the development of a cancer vaccine associated with the specific marker cell surface antigenic determinant introduced into the cancer specific and CIN associated recombinant events identified within the cancer cells that are not present in the normal healthy cells. Once treated with the cancer specific CRISPR/Cas9 complexes, and the expression of the marker cell surface antigen on the cancer cells, a single vaccine treatment inducing a specific immune reaction by the patient would elicit an immune reaction to the cancer cells in addition to the viral infection. Currently, a similar immune therapeutic approach has been successfully demonstrated in the clinic, but requires the purification of cancer cells and the identification of an endogenous cancer specific cell surface antigen or antigens. Utilization of CRISPR/Cas9 complexes to target and express a marker cell surface antigen onto the cell surface of cancer cells takes advantage of the unique and universal feature of Chromosome Instability associated with neoplasia, and would not limit treatment to a limited population of cancer specific patient populations.

A fourth embodiment disclosed involves the introduction of a mixture of CRISPR/Cas9 complexes whereby each fusion sequence present in the cells of pre-cancerous or cancerous cells and not present in normal cells has a pair of CRISPR/Cas9 complexes directed to each of several cancer specific fusion sequence. Within each pair of CRISPR/Cas9 complexes targeting a single fusion sequence, one of the CRISPR/Cas9 complexes would contain a guide RNA complementary to one half of the fusion sequence, and the second CRISPR/Cas9 complex would contain a second unique sequence complementary to the sequence immediately adjacent to the first guide RNA associated with the first CRISPR/Cas9 complex. One embodiment of the composition disclosed includes two or more CRISPR/Cas9 complexes that recognize to two separate regions of a cell's genomic DNA that are distant from one another in a healthy cell. One of the CRISPR/Cas9 complexes contains a cytotoxic agent and the other contains an activator of the cytotoxic agent. The activator activates the cytotoxic agent only when the two CRISPR/Cas9 complexes hybridize to regions of the genome that are within proximity sufficient for the activation to occur. The CRISPR/Cas9 complexes are designed to 1) recognize regions of the target genome that are separated in a healthy cell by a distance that is too great for the activator to induce the cytotoxic agent upon hybridization of the CRISPR/Cas9 complexes and 2) hybridize to regions that are sufficiently close for cytotoxic activation in a cell that is genomically-unstable. The design of the CRISPR/Cas9 complexes is preferably, but not necessarily, driven by sequencing nucleic acid in cancer cells (e.g., cells from a biopsy) to determine where genomic instability (e.g., a deletion) has occurred.

The disclosure also contemplates methods comprising administering a first CRISPR/Cas9 complex and a second CRISPR/Cas9 complexes to a subject. A first CRISPR/Cas9 complex comprises a cytotoxic agent and the second CRISPR/Cas9 complex comprises an activator of the cytotoxic agent. For example, an activating agent is attached to the CRISPR/Cas9 complexes, and a prodrug of a chemotherapeutic agent is attached to a CRISPR/Cas9 complex. The CRISPR/Cas9 complexes are designed to hybridize to first and second sequence portions that are identical in the wild-type sequence and the mutated sequence. The first and second CRISPR/Cas9 complexes flank the region of genetic material that is lost from a wild-type sequence to result in the mutated sequence present in the cancerous and pre-cancerous cells. Thus, the CRISPR/Cas9 complexes are brought into proximity for activation of the therapeutic agent only when there is a loss of genomic material. While the probes can hybridize to contiguous regions in the mutated cells, all that is required is that they hybridize in sufficient proximity for activation of the cytotoxic agent in the mutated cells (but are out of proximity for activation in a healthy cell).

Upon administration of the CRISPR/Cas9 complexes to a subject, the first and second CRISPR/Cas9 complexes hybridize to the first and second portions of the sequences in the normal cells and in the cancerous or pre-cancerous cells. For the wild-type sequences, the first and second portions are not within sufficient proximity of each other for the activating agent to convert the prodrug to an active form of the chemotherapeutic agent. Thus the chemotherapeutic agent remains inactive and the normal cell is unharmed.

However, the sequences in the cancerous or pre-cancerous cells have undergone a mutation resulting in loss of a certain amount of genetic material between the first and second portions. Thus in the mutated sequences, the first and second portions are within sufficient proximity of each other for the activating agent to convert the prodrug to an active form of the chemotherapeutic agent, thereby providing targeted delivery of the chemotherapeutic agent to the cancerous or pre-cancerous cell in the subject, and killing those cells.

One aspect of the embodiment provides a method of treating a cancer including administering to a subject a prodrug of a chemotherapeutic agent, coupled to a first CRISPR/Cas9 complex, and administering an activating agent, coupled to a second CRISPR/Cas9 complex, in which the complexes hybridize to a first sequence portion and a second sequence portion that are identical in both a wild-type sequence found in a normal cell of the subject and a mutated sequence found in a cancerous or pre-cancerous cell of the subject. In the wild-type sequence, the first and second portions are not within sufficient proximity to each other for the activating agent to convert the prodrug to an active form of the chemotherapeutic agent. In the mutated sequence, the first and second portions are within sufficient proximity to each other for the activating agent to convert the prodrug to an active form of the chemotherapeutic agent, thereby providing targeted delivery of the chemotherapeutic agent to the cancerous or pre-cancerous cell in the subject.

One aspect of the disclosure provides a method of treating a cancer including administering to a subject a CRISPR/Cas9 complex or mixture of CRISPR/Cas9 complexes complementary to cancer specific recombination events resulting from chromosome instability (“CIN”) known to be associated with malignancy, that do not occur in normal healthy cells.

Differences in sequences between cancer/pre-cancer and normal/healthy cells may be determined by many methods, such as sequencing. Sequencing may be by a chain-termination sequencing technique (Sanger sequencing) or by a single molecule sequencing-by-synthesis technique. In certain embodiments, a nucleic acid is obtained from the normal cell of the subject and sequenced, thereby acquiring a wild-type sequence. Also, a nucleic acid is obtained from the cancerous or pre-cancerous cell of the same subject and sequenced, thereby acquiring a mutated sequence. Once the two different sequences are acquired, the wild-type sequence and the mutated sequences may be compared, and thus a determination of the difference between the wild-type sequence and the mutated sequence is made. The difference between the wild-type sequence and the mutated sequence is the mutated regions to which the CRISPR/Cas9 complexes will be designed. In certain embodiments, the difference between the wild-type sequence and the mutated sequence is the result of a loss of genetic material between the first and second portions in the mutated sequence, in which the loss of genetic material results from a mutation event including a deletion, a substitution, or a rearrangement.

The compositions and methods disclosed may be used to treat any cancer. Examples of such cancers include brain, bladder, blood, bone, breast, cervical, colorectal, gastrointestinal, endocrine, kidney, liver, lung, ovarian, pancreatic, prostate, and thyroid.

Another aspect of the disclosure provides a method of treating a cancer in a subject including sequencing a nucleic acid found in a normal cell of a subject to obtain a wild-type sequence. The method further involves sequencing a nucleic acid found in a cancerous or pre-cancerous cell of the same subject, to obtain a mutated sequence of the cancerous or pre-cancerous cell of the subject. Once both sequences have been obtained, the wild-type sequence and the mutated sequence may be compared which results in a determination of the difference between the two sequences, correlating to the difference in sequences between a normal cell and a cancerous or pre-cancerous cell of the subject.

After determining the difference, the methods further involve administering to the subject a CRISPR/Cas9 complex or mixture of CRISPR/Cas9 complexes each having a guide RNA specific to the fusion sequences identified within the cancer or pre-cancerous cell that is not present in the sequence of the normal cells. Upon recognition and binding of the CRISPR/Cas9 complex or complexes to the cancer specific fusion sequences complementary to the guide RNA sequences, the CRISPR/Cas9 complex or complexes will cut the genomic DNA at their respective sites and initiate cell death, introduce a gene sequence coding for a protein product lethal to the cancer cells, or introduce a gene sequence that codes for a marker cell surface antigen specific to the cancer cells and not present in normal healthy cells.

Use of various Cas endonuclease complexes may provide different advantages, for example, a Cas13a endonuclease (in contrast to Cas9) is capable of cleaving RNA, does not require a PAM sequence at the target locus, and may display a higher specificity compared to other Cas endonucleases. A CRISPR/Cas13a complex, may be created using similar methods to the CRISPR/Cas9 complex, but may be used to target specific regions of RNA. Multiple Cas13a complexes or a mixture of Cas13a complexes may be used. For example, upon administration of the CRISPR/Cas13a complex to a subject, the CRISPR/Cas13a complexes each target their respective fusion sequences and create several single strand cuts within the RNA, therefore inducing cell death within the cancer cells. However, due to the lack of homology within the normal healthy cells, the CRISPR/Cas13a complexes do not interact with RNA of healthy normal cells, and those normal cells are unharmed.

Compositions include a Cas endonuclease or nucleic acid encoding the Cas endonuclease and a guide RNA that targets the Cas endonuclease to a fusion sequence that is in a cancer cell but not in a healthy cell of the subject. The guide RNA may contain a targeting sequence that is complementary to the fusion sequence. The targeting sequence of the guide RNA may be assembled complementary to the fusion sequence based on a difference identified between a mutated sequence obtained from sequencing the cancer cell and a wild-type sequence obtained from sequencing the healthy cell. To identify such differences, sequencing may be performed by any suitable sequencing technique. For example, sequencing may be performed by a single molecule sequencing-by-synthesis technique. In one embodiment, the Cas endonuclease is a Cas9 endonuclease that cuts DNA. In another embodiment, the Cas endonuclease is a Cas13a endonuclease that cuts RNA.

In one embodiment, to treat cancer, the Cas endonuclease induces cell death by generating a strand break in the fusion sequence. In another embodiment, the Cas endonuclease induces cell death by incorporating a protein coding gene sequence that results in expression of a lethal protein. In yet another embodiment, the Cas endonuclease induces expression of a marker cell surface protein by incorporating a protein coding gene that when expressed results in the marker cell surface protein.

The cancer cell of the subject may include an aneuploidy. For example, the aneuploidy may be any of an inversion, a deletion, a loss of heterozygosity, and a genetic rearrangement. The cancer may be any of brain, bladder, blood, bone, breast, cervical, colorectal, gastrointestinal, endocrine, kidney, liver, lung, ovarian, pancreatic, prostate, or thyroid.

Methods for treating cancer include administering to a subject a Cas endonuclease or nucleic acid encoding the Cas endonuclease and a guide RNA that targets the Cas endonuclease to a fusion sequence that is in a cancer cell but not in a healthy cell of the subject. In one embodiment, the method further includes sequencing nucleic acid from the cancer cell to obtain a mutated sequence and sequencing nucleic acid from the healthy cell to obtain a wild-type sequence. The method may further include designing the guide RNA to target the Cas endonuclease to the fusion sequence by identifying the fusion sequence based on a difference between the wild-type sequence and the mutated sequence. In one example, sequencing is performed by a single molecule sequencing-by-synthesis technique.

To treat cancer, in various embodiments, the Cas endonuclease may be delivered as a protein complexed with the guide RNA, delivered as a DNA that encodes the Cas endonuclease to be transcribed in cells of the subject, or delivered as an mRNA to be translated in cells of the subject. In such embodiments, the guide RNA contains a targeting sequence that is complementary to the fusion sequence. The Cas endonuclease may be, for example, a Cas9 endonuclease that cuts DNA or a Cas13a endonuclease that cuts RNA. To treat cancer, in one embodiment, the Cas endonuclease induces cell death by generating a strand break in the fusion sequence. In another embodiment, the Cas endonuclease induces cell death by incorporating a protein coding gene sequence that results in expression of a lethal protein. In yet another embodiment, the Cas endonuclease or induces expression of a marker cell surface protein by incorporating a protein coding gene that when expressed results in the marker cell surface protein.

The cancer cell of the subject may include an aneuploidy. For example, the aneuploidy may be any of an inversion, a deletion, a loss of heterozygosity, and a genetic rearrangement. The cancer may be any of brain, bladder, blood, bone, breast, cervical, colorectal, gastrointestinal, endocrine, kidney, liver, lung, ovarian, pancreatic, prostate, or thyroid.

### DETAILED DESCRIPTION

Many cancers are thought to arise through a series of mutations in genomic DNA, resulting in genomic instability in the form of uncontrolled cellular growth. In normal cells, damage to genomic DNA typically leads to expression of tumor suppressors, such as the cell-cycle regulator, p53. For example, damage to cellular DNA results in increased expression of p53 which arrests the cell cycle to allow repair of the damage. If the damaged DNA cannot be repaired, the cell undergoes apoptosis, thus preventing the accumulation of additional mutations in daughter cells. If however, there is a mutation in the p53 gene itself (or in another cell cycle regulator), damaged cells will proceed through the cell cycle, giving rise to progeny in which additional DNA mutations will go unchecked. It is the accumulation of these mutations that is the hallmark of many cancers.

The disclosure generally relates to compositions and methods for targeted delivery of a Cas endonuclease or nucleic acid encoding the Cas endonuclease to cancerous and pre-cancerous cells, thereby treating a cancer in a subject. Disclosed methods involve administering CRISPR/Cas9 complexes that hybridize to unique fusion sequences present in cancer cells due to the mechanism of chromosome instability that do not exist in wild type sequences present in normal or healthy cells. A wild-type sequence from a normal cell is that which is most frequently observed in a population and is thus arbitrarily designed the “normal” or “wild-type” sequence.

In contrast, the abnormal or mutant sequence refers to a sequence that displays modifications in sequence and or functional properties (i.e., altered characteristics) when compared to the wild-type sequence. For example, an altered sequence detected in the urine or plasma of a patient can display a modification that occurs in DNA sequences from tumor cells and that does not occur in the patient's normal (i.e. non cancerous) cells. It is noted that naturally-occurring mutants can be isolated; these are identified by the fact that they have altered characteristics when compared to the wild-type gene or gene product.

The disclosure should not be limited to detection of any specific type of anomaly because mutations can take many forms. A common genetic change characteristic of transformation is loss of heterozygosity. Loss of heterozygosity at a number of tumor suppressor genes has been implicated in tumorigenesis. For example, loss of heterozygosity at the P53 tumor suppressor locus has been correlated with various types of cancer. Ridanpaa, et al., Path. Res. Pract, 191; 399-402 (1995), incorporated by reference. The loss of the apc and dcc tumor suppressor genes has also been associated with tumor development. Blum, Europ. J. Cancer, 31A: 1369-372 (1995), incorporated by reference.

Certain mutations that result in loss of genetic material giving rise to cancer and the locations within a gene of those mutations may be published. See, e.g., Hesketh, The Oncogene Facts Book, Academic Press Limited (1995), incorporated by reference. By knowing the mutation and the location of the mutation, a CRISPR/Cas9 complex may be designed that will recognize only mutated fusion sequences present in the cancer cells and not present in the normal healthy cells.

Alternatively, samples from the subject may be obtained and sequenced in order to determine the differences between the wild-type sequences from normal cells and the mutant sequences from cancerous and pre-cancerous cells.

To obtain the wild-type and mutant sequences, a sample is obtained from a subject. The sample may be obtained in any clinically acceptable manner, and the nucleic acids may be extracted from the sample by any suitable method. Generally, nucleic acid can be extracted from a biological sample by a variety of techniques such as those described by Maniatis, et al. (Molecular Cloning: A Laboratory Manual, Cold Spring Harbor, N.Y., pp. 280-281, 1982), incorporated by reference.

The sample may be a human tissue or bodily fluid. A tissue is a mass of connected cells and/or extracellular matrix material, e.g. skin tissue, nasal passage tissue, CNS tissue, neural tissue, eye tissue, liver tissue, kidney tissue, placental tissue, mammary gland tissue, placental tissue, gastrointestinal tissue, musculoskeletal tissue, genitourinary tissue, bone marrow, and the like, derived from, for example, a human or other mammal and includes the connecting material and the liquid material in association with the cells and/or tissues.

A bodily fluid is a liquid material derived from, for example, a human or other mammal. Such body fluids include, but are not limited to, mucous, blood, plasma, serum, serum derivatives, bile, blood, maternal blood, phlegm, saliva, sweat, amniotic fluid, mammary fluid, urine, and cerebrospinal fluid (CSF), such as lumbar or ventricular CSF. A sample may also be a fine needle aspirate or biopsied tissue. A sample also may be media containing cells or biological material. In certain embodiments, the sample includes nucleic acid molecules that are cell free circulating nucleic acid molecules.

Once obtained, the nucleic acid molecules may be sequenced by any of various methods, for example, ensemble sequencing or single molecule sequencing. One conventional method to perform sequencing is by chain termination and gel separation, as described by Sanger et al., Proc Natl Acad Sci U S A, 74(12):5463 67 (1977), incorporated by reference. Another conventional sequencing method involves chemical degradation of nucleic acid fragments. See, Maxam et al., Proc. Natl. Acad. Sci., 74:560 564 (1977), incorporated by reference. Finally, methods have been developed based upon sequencing by hybridization. See, e.g., Drmanac, et al. (Nature Biotech., 16:54 58, 1998), incorporated by reference.

In certain embodiments, sequencing may be performed by the Sanger sequencing technique. Classical Sanger sequencing involves a single-stranded DNA template, a DNA primer, a DNA polymerase, radioactively or fluorescently labeled nucleotides, and modified nucleotides that terminate DNA strand elongation. If the label is not attached to the dideoxynucleotide terminator (e.g., labeled primer), or is a monochromatic label (e.g., radioisotope), then the DNA sample is divided into four separate sequencing reactions, containing four standard deoxynucleotides (dATP, dGTP, dCTP and dTTP) and the DNA polymerase. To each reaction is added only one of the four dideoxynucleotides (ddATP, ddGTP, ddCTP, or ddTTP), These dideoxynucleotides are the chain-terminating nucleotides, lacking a 3′-OH group required for the formation of a phosphodiester bond between two nucleotides during DNA strand elongation. If each of the dideoxynucleotides carries a different label, however, (e.g., 4 different fluorescent dyes), then all the sequencing reactions can be carried out together without the need for separate reactions.

Incorporation of a dideoxynucleotide into the nascent (i.e., elongating) DNA strand terminates DNA strand extension, resulting in a nested set of DNA fragments of varying length.

Newly synthesized and labeled DNA fragments are denatured, and separated by size using gel electrophoresis on a denaturing polyacrylamide-urea gel capable of resolving single-base differences in chain length. If each of the four DNA synthesis reactions was labeled with the same, monochromatic label (e.g., radioisotope), then they are separated in one of four individual, adjacent lanes in the gel, in which each lane in the gel is designated according to the dideoxynucleotide used in the respective reaction, i.e., gel lanes A, T, G, C. If four different labels were utilized, then the reactions can be combined in a single lane on the gel. DNA bands are then visualized by autoradiography or fluorescence, and the DNA sequence can be directly read from the X-ray film or gel image.

The terminal nucleotide base is identified according to the dideoxynucleotide that was added in the reaction resulting in that band or its corresponding direct label. The relative positions of the different bands in the gel are then used to read (from shortest to longest) the DNA sequence as indicated. The Sanger sequencing process can be automated using a DNA sequencer, such as those commercially available from PerkinElmer, Beckman Coulter, Life Technologies, and others.

In other embodiments, sequencing of the nucleic acid may be accomplished by a single-molecule sequencing by synthesis technique. Single molecule sequencing is shown for example in Lapidus et al. (U.S. Pat. No. 7,169,560), Quake et al. (U.S. Pat. No. 6,818,395), Harris (U.S. Pat. No. 7,282,337), Quake et al. (U.S. patent application number 2002/0164629), and Braslavsky, et al., PNAS (USA), 100:3960-3964 (2003), each of which are incorporated by reference. Briefly, a single-stranded nucleic acid (e.g., DNA or cDNA) is hybridized to oligonucleotides attached to a surface of a flow cell. The oligonucleotides may be covalently attached to the surface or various attachments other than covalent linking may be employed. Moreover, the attachment may be indirect, e.g., via a polymerase directly or indirectly attached to the surface. The surface may be planar or otherwise, and/or may be porous or non-porous, or any other type of surface suitable for attachment. The nucleic acid is then sequenced by imaging the polymerase-mediated addition of fluorescently-labeled nucleotides incorporated into the growing strand surface oligonucleotide, at single molecule resolution.

Other single molecule sequencing techniques involve detection of pyrophosphate as it is cleaved from incorporation of a single nucleotide into a nascent strand of DNA, as is shown in Rothberg et al. (U.S. Pat. Nos. 7,335,762, 7,264,929, 7,244,559, and 7,211,390) and Leamon et al. (U.S. Pat. No. 7,323,305), each of which is incorporated by reference.

In other embodiments, targeted resequencing is used. Resequencing is shown for example in Harris (U.S. patent application numbers 2008/0233575, 2009/0075252, and 2009/0197257), each of which is incorporated by reference. Briefly, a specific segment of the target is selected (for example by PCR, microarray, or MIPS) prior to sequencing. A primer designed to hybridize to this particular segment, is introduced and a primer/template duplex is formed. The primer/template duplex is exposed to a polymerase, and at least one detectably labeled nucleotide under conditions sufficient for template dependent nucleotide addition to the primer. The incorporation of the labeled nucleotide is determined, as well the identity of the nucleotide that is complementary to a nucleotide on the template at a position that is opposite the incorporated nucleotide.

After the polymerization reaction, the primer may be removed from the duplex. The primer may be removed by any suitable means, for example by raising the temperature of the surface or substrate such that the duplex is melted, or by changing the buffer conditions to destabilize the duplex, or combination thereof. Methods for melting template/primer duplexes are described, for example, in chapter 10 of Molecular Cloning, a Laboratory Manual, 3.sup.rd Edition, J. Sambrook, and D. W. Russell, Cold Spring Harbor Press (2001), incorporated herein by reference.

After removing the primer, the template may be exposed to a second primer capable of hybridizing to the template. In one embodiment, the second primer is capable of hybridizing to the same region of the template as the first primer (also referred to herein as a first region), to form a template/primer duplex. The polymerization reaction is then repeated, thereby resequencing at least a portion of the template.

If the nucleic acid from the sample is degraded or only a minimal amount of nucleic acid can be obtained from the sample, PCR can be performed on the nucleic acid in order to obtain a sufficient amount of nucleic acid for sequencing (See e.g., Mullis et al. U.S. Pat. No. 4,683,195, incorporated by reference).

Once the wild-type sequences from the normal cells and the mutant sequences from the cancerous or pre-cancerous cells are obtained, these sequences are compared to determine the differences between the sequences. The difference of interest is a loss of genetic material from the wild-type sequence, e.g., a deletion event, that results in the mutant sequence found in the cancerous or pre-cancerous cells.

After determining the region of genetic material that is lost from the wild-type sequence to result in the mutant sequence, the regions of the sequences that flank the mutated region in both the wild-type and mutant sequences (i.e., sequences upstream of the mutated region and downstream of the mutated region) are analyzed. Based on the analysis of the sequences that flank the mutated region, CRISPR/Cas9 complexes and guide RNA molecules are designed to hybridize and target the fusion sequences unique to the cancer and pre-cancerous DNA, and are not present in the normal and healthy cellular DNA.

Upon administration of the CRISPR/Cas9 complexes to a subject, the CRISPR/Cas9 complexes may only interact with the fusion specific sequences within the cancerous or pre-cancerous DNA. For the wild-type sequences, the unique cancer specific fusion sequences are not present and therefore the CRISPR/Cas9 complexes do not hybridize and interact with the genomic sequences

However, the sequences in the cancerous or pre-cancerous cells have undergone the mutation event resulting in loss of genetic material between the first and second portions. Thus in the mutated sequences, the CRISPR/Cas9 complexes hybridize and may either cut at the sequence of homology, or may introduce a lethal protein coding or marker cell surface antigen coding sequence into the cancer associated mutant DNA.

The disclosed compositions may be administered using any amount and any route of administration effective for treating the cancer. Thus, the expression “amount effective for treating a cancer”, as used herein, refers to a sufficient amount of composition to beneficially prevent or ameliorate the symptoms of the cancer.

The exact dosage may be chosen by an individual physician in view of the patient to be treated and certain other factors. Dosage and administration are adjusted to provide sufficient levels of the CRISPR/Cas9 complexes or to maintain the desired effect. Additional factors which may be taken into account include the severity of the disease state, age, weight and gender of the patient; diet, time and frequency of administration; route of administration; drug combinations; reaction sensitivities; and tolerance/response to therapy. Long acting pharmaceutical compositions might be administered, for example, hourly, twice hourly, every 3 to four hours, daily, twice daily, every 3 to 4 days, every week, or once every two weeks depending on half-life and clearance rate of the particular composition.

The disclosed CRISPR/Cas9 complexes or mixture of CRISPR/Cas9 complexes may preferably be formulated in dosage unit form for ease of administration and uniformity of dosage. The expression “dosage unit form” as used herein refers to a physically discrete unit of CRISPR/Cas9 complexes appropriate for the patient to be treated. It will be understood, however, that the total daily usage of the disclosed compositions will be decided by the attending physician within the scope of sound medical judgment. For any active agent, the therapeutically effective dose may be estimated initially either in cell culture assays or in animal models, as provided herein, usually mice, but also potentially from rats, rabbits, dogs, or pigs. The animal cell model provided herein is also used to achieve a desirable concentration and total dosing range and route of administration. Such information can then be used to determine useful doses and routes for administration in humans.

A therapeutically effective dose refers to that amount of CRISPR/Cas9 complexes that ameliorates the symptoms or condition or prevents progression of the cancer. Therapeutic efficacy and toxicity of active agents can be determined by standard pharmaceutical procedures in cell cultures or experimental animals. For example, therapeutic efficacy and toxicity can be determined by minimal efficacious dose or NOAEL (no observable adverse effect level). Alternatively, an ED50 (the dose is therapeutically effective in 50% of the population) and LD50 (the dose is lethal to 50% of the population) can be determined in cell cultures or experimental animals. The dose ratio of toxic to therapeutic effects is the therapeutic index, and it can be expressed as the ratio, LD50/ED50. Pharmaceutical compositions that exhibit large therapeutic indices are preferred.

As formulated with an appropriate pharmaceutically acceptable carrier in a desired dosage, the pharmaceutical composition provided herein is administered to humans and other mammals topically such as ocularly, nasally, bucally, orally, rectally, parenterally, intracisternally, intravaginally, or intraperitoneally.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, PA, 1995, incorporated by reference, provides various carriers used in formulating pharmaceutical compositions and techniques for the preparation thereof. Some examples of materials which can serve as pharmaceutically acceptable carriers include, but are not limited to, sugars such as glucose and sucrose; excipients such as cocoa butter and suppository waxes; oils such as peanut oil, cottonseed oil, safflower oil, sesame oil, olive oil, corn oil, and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, preservatives and antioxidants can also be present in the composition, according to the judgment of the formulator.

Liquid dosage forms for ocular, oral, or other systemic administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the ocular, oral, or other systemically-delivered compositions can also include adjuvants such as wetting agents, and emulsifying and suspending agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. For example, ocular or cutaneous routes of administration are achieved with aqueous drops, a mist, an emulsion, or a cream. Administration may be therapeutic or it may be prophylactic. The disclosure includes ophthalmological devices, surgical devices, audiological devices or products which contain disclosed compositions (e.g., gauze bandages or strips), and methods of making or using such devices or products. These devices may be coated with, impregnated with, bonded to or otherwise treated with a composition as described herein.

Transdermal patches have the added advantage of providing controlled delivery of the active ingredients to the body. Such dosage forms may be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Compositions for rectal or vaginal administration are preferably suppositories which can be prepared by mixing the active agent(s) of this invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings suitable for pharmaceutical formulation. In such solid dosage forms the active agent(s) may be admixed with at least one inert diluent such as sucrose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain pacifying agents and can also be of a composition that they release the active agent(s) only, or preferentially, in a certain part of the intestinal tract, optionally, in a delayed manner. Examples of embedding compositions which can be used include polymeric substances and waxes.

This disclosure provides compositions and methods for the targeted delivery of gene editing systems to a fusion sequence present in a cancer cell or pre-cancerous cell to treat cancer in a subject. In preferred embodiments, the gene editing system comprises a CRISPR associated protein complexed with guide RNA. The genome-editing system may be, for example, a Cas endonuclease complexed with guide RNA. The Cas endonuclease may be, for example, Cas9 (e.g., spCas9), Cpf1 (aka Cas12a), C2c2, Cas13, Cas13a, Cas13b, e.g., PsmCas13b, LbaCas13a, LwaCas13a, AsCas12a, PfAgo, NgAgo, CasX, CasY, others, modified variants thereof, and similar proteins or macromolecular complexes. In other embodiments, the gene editing system may include at least one transcription activator-like effector nuclease (TALEN) with a primary amino acid sequence that confers target specificity on the TALEN to a target (e.g., a fusion) in the genome of tumor cell in a subject. TALENs are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to practically any desired DNA sequence (e.g., fusion sequences), so when combined with a nuclease, DNA can be cut at specific locations.

In other embodiments, the genome-editing system is a zinc-finger nuclease. Zinc-finger nucleases are artificial restriction enzymes generated by fusing a zinc finger DNA-binding domain to a DNA-cleavage domain. Zinc finger domains can be engineered to target specific desired DNA sequences such as fusion sequence.

In other embodiments, the gene editing system may comprise a meganuclease. Meganucleases are endodeoxyribonucleases characterized by a large recognition site (double-stranded DNA sequences of 12 to 40 base pairs).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for treating cancer, the method comprising:
administering to a subject a Cas endonuclease or nucleic acid encoding the Cas endonuclease and a guide RNA that targets the Cas endonuclease to a fusion sequence that is in a cancer cell but not in a healthy cell of the subject.

2. The method of claim 1, further comprising:
sequencing nucleic acid from the cancer cell to obtain a mutated sequence and sequencing nucleic acid from the healthy cell to obtain a wild-type sequence; and
designing the guide RNA to target the Cas endonuclease to the fusion sequence by identifying the fusion sequence based on a difference between the wild-type sequence and the mutated sequence.

3. The method of claim 1, wherein the guide RNA contains a targeting sequence that is complementary to the fusion sequence.

4. The method of claim 1, wherein the Cas endonuclease is a Cas9 endonuclease that cuts DNA or a Cas13a endonuclease that cuts RNA.

5. The method of claim 1, wherein the Cas endonuclease is delivered as a protein complexed with the guide RNA.

6. The method of claim 1, wherein the Cas endonuclease induces cell death by generating a strand break in the fusion sequence or by incorporating a protein coding gene sequence that results in expression of a lethal protein.

7. The method of claim 1, wherein the Cas endonuclease induces expression of a marker cell surface protein by incorporating a protein coding gene that when expressed results in the marker cell surface protein.

8. The method of claim 1, wherein the cancer cell comprises an aneuploidy, the aneuploidy selected from a group consisting of an inversion, a deletion, a loss of heterozygosity, and a genetic rearrangement.

9. A composition for the treatment of cancer, the composition comprising:
a Cas endonuclease or nucleic acid encoding the Cas endonuclease and a guide RNA that targets the Cas endonuclease to a fusion sequence that is in a cancer cell but not in a healthy cell of the subject.

10. The composition of claim 9, wherein the guide RNA contains a targeting sequence that is complementary to the fusion sequence.

11. The composition of claim 10, wherein the targeting sequence of the guide RNA is assembled complementary to the fusion sequence based on a difference identified between a mutated sequence obtained from sequencing the cancer cell and a wild-type sequence obtained from sequencing the healthy cell.

12. The composition of claim 9, wherein the Cas endonuclease is a Cas9 endonuclease that cuts DNA or a Cas13a endonuclease that cuts RNA.

13. The composition of claim 9, wherein the Cas endonuclease induces cell death by generating a strand break in the fusion sequence or by incorporating a protein coding gene sequence that results in expression of a lethal protein.

14. The composition of claim 9, wherein the Cas endonuclease induces expression of a marker cell surface protein by incorporating a protein coding gene that when expressed results in the marker cell surface protein.

15. The composition of claim 9, the cancer cell comprises an aneuploidy, the aneuploidy is selected from a group consisting of an inversion, a deletion, a loss of heterozygosity, and a genetic rearrangement.

16. The composition of claim 9, wherein the cancer is selected from a group consisting of brain, bladder, blood, bone, breast, cervical, colorectal, gastrointestinal, endocrine, kidney, liver, lung, ovarian, pancreatic, prostate, and thyroid.

17. A method for treating a cancer in a subject, the method comprising:
sequencing a nucleic acid found in a normal cell of a subject, thereby obtaining a wild-type sequence;
sequencing a nucleic acid found in a cancerous or pre-cancerous cell of the subject, thereby obtaining a mutated sequence;
comparing the wild-type sequence and the mutated sequence, thereby determining a difference between the wild-type sequence and the mutated sequence; and
administering to the subject a single CRISPR/Cas9 complex or a mixture of CRISPR/Cas9 complexes whose guide RNA hybridize to fusion sequences of the genome that are in a cancer cell but not in a healthy cell.

18. The method according to claim 17, wherein a single CRISPR/Cas9 complex or a mixture of CRISPR/Cas9 complexes target cancer specific fusion sequences and generate double strand breaks inducing cell death.

19. The method according to claim 18, wherein a single CRISPR/Cas9 complex or a mixture of CRISPR/Cas9 complexes target cancer specific fusion sequences and incorporate a protein coding gene sequence that results in the expression of a lethal protein and induces cell death.

20. The method according to claim 18, wherein a single CRISPR/Cas9 complex or a mixture of CRISPR/Cas9 complexes target cancer specific fusion sequences and incorporate a protein coding gene sequence that results in the expression of a protein that becomes expressed and represents a marker cell surface protein.
